(19) United States
(12) Patent Application Publication
(10) Pub. No.: US 2010/0121037 A1
(43) Pub. Date: May 13, 2010

(54) METHOD FOR SELECTIVE FRACTIONATION OF GROWTH FACTORS FROM DAIRY PRODUCTS

(75) Inventors: David Lachkar, Quebec (CA); Yves Pouliot, Charny (CA); Sylvie Gauthier, Charny (CA); Sylvie Turgeon, L'Ancienne-Lorette (CA); Eric Lamiot, rue Desroches (CA); Christina Juneau, Stoneham-et-Tewkesbury (CA)

Correspondence Address: SUTHERLAND ASBILL & BRENNAN LLP, 999 PEACHTREE STREET, N.E., ATLANTA, GA, 30309, US

(73) Assignee: UNIVERSITE LAVAL, QC, QC (CA)

(21) Appl. No.: 12/594,528

(22) PCT Filed: Apr. 1, 2008
(86) PCT No.: PCT/CA08/00628

§ 371 Date: Jan. 11, 2010

Related U.S. Application Data
(60) Provisional application No. 60/909,564, filed on Apr. 2, 2007.

(51) Int. Cl. C07K 1/14

(52) U.S. Cl. 530/399

(57) ABSTRACT

The invention relates to the general field of protein fractionation, and more particularly to the selective fractionation of growth factor from dairy products, such as milk, whey and colostrum. There is provided methods for the selective fractionation of TGF-β, lactoferrin and bovine serum albumin.

### FIELD OF THE INVENTION

The present invention relates in general to a method for the selective fractionation of growth factors from dairy products, such as milk, whey and colostrum.

### BACKGROUND OF THE INVENTION

Milk is an emulsion of fat globules within a water-based fluid. While raw milk contains fat globules, carbohydrates, blood cells, mammary gland cells, bacteria and enzymes, the largest structures found in the fluid portion of the milk are casein protein micelles, which are roughly an aggregate of proteins bonded together with calcium phosphate. Caseins are the most predominant phosphoproteins in milk, and they are not coagulated by heat but rather precipitated by rennet enzymes or by acids. They consist of proline-rich peptides that do not interact with one another, and do not have any disulfure bonds. The different types of caseins represents about 80% of the proteins in milk. The other 20% is mostly made of water-soluble proteins, such as lactoglobulins, which are usually referred to as whey proteins since they remain in whey when casein coagulates into curds.

Colostrum is the first nutrient produced by the mammary glands of a female at the end of pregnancy and in the first days after giving birth. It is a complex fluid that is rich in nutriments, antibodies and growth factors, and its composition varies greatly and rapidly in the few days it is produced:

| | Initial | Concentration |
|---|---|---|
| Component | concentration (%) | 72 h after birth (%) |
| Total solids | 23.9 | 13.6 |
| Proteins | 14 | 4.1 |
| Caseins | 4.8 | 2.9 |
| Fat | 6.7 | 4.3 |
| Lactose | 2.7 | 4.7 |
| Mineral salts | 1.11 | 0.81 |

In addition to nutriments, such as proteins, carbohydrates, fat, vitamins and minerals, colostrum also contains many bioactive molecules having essential functions. Those bioactive molecules are found in the protein fraction of the colostrum, such as albumins. Albumins, such as bovine serum albumin (BSA) for example, are hydrosoluble proteins that mainly serves a role of transport proteins in organisms. They are essential for maintaining the osmotic pressure needed for the correct distribution of body fluids between intravascular compartments and body tissues. Amongst the more important bioactive molecules are growth factors and antimicrobial factors. Growth factors allow for the growth and development of the newborn while the antimicrobial factors protect the newborn from infections during the first weeks of life. The antimicrobial activity of the colostrum is essentially performed by immunoglobulins, with a minimal contribution by lactoferrin, lysozyme and lactoperoxidase.

Lactoferrin (LF) is a globular multifunctional protein having an antimicrobial activity that is often referred to as an innate defense protein. Many potential physiological roles have been attributed to LF, such as anti-bacterial, anti-fungal, anti-viral, antioxidant and immunomodulator.

Of the growth factors present in milk (insulin-like growth factors (IGF), IGF-binding proteins, epidermal growth factor (EGF), transforming growth factor (TGF), etc), many of them are present in concentration exceeding those found in maternal plasma. This particularity makes milk and milk-derived products a great source for extracting and purifying growth factors.

Transforming growth factor β (TGF-β) is a family of growth factors regulating cell growth and differentiation. In order to extract TGF-β from bovine colostrum, it is necessary to extract caseins prior to obtain a serocolostrum.

Various processes have been tested and applied in the art for extracting nutrients from dairy products. Apart from the most commonly used techniques of rennet or acidic precipitation, other processes have been proposed for the precipitation of caseins, such as chemical processes using polysaccharides, ethanol or calcium chloride, or physical processes such as microfiltration or cryoprecipitation.

Acidic precipitation. Acidification of milk mostly affects milk caseins. Even a slight acidification of milk pH induces a sufficient modification of micelles structures for the caseins to become unstable to heat. Usually, acidification is performed with HCl. Thus, when milk pH changes from 6.7 to 5.5, there is a neutralization of the negative charges present on the surface of casein micelles, which leads to the aggregation of smaller micelles into micelles of greater diameter. The aggregation is such that the micelles keep their spherical shape, and essentially produces micelles having a generally increased diameter. When acidification continues up to pH 5.0, aggregation continues and one can even note fusion between some of the micelles. When the isoelectric point of caseins is reached, which is at pH 4.65, the calcium content of the micelles dissolves, leading to the denaturation of caseins and to the loss of their colloidal suspension ability. Caseins are then submitted to a stretching that can lead to their entanglement and the formation of a gel-like structure.

Rennet precipitation. Rennet is a natural complex of enzymes produced by mammalian stomach to digest the mother's milk, mainly composed of proteolytic enzymes such as chymosin, pepsin and lipase. Adding rennet to milk causes coagulation of milk, separating milk into solids (curds) and liquid (whey). Coagulation follows the enzymatic hydrolysis of κ-casein present on the periphery of the micelle. This hydrolysis occurs between Phe¹⁰⁵ and Met¹⁰⁶ of the κ-casein, and releases a glycomacropeptide corresponding to the hydrophilic portion of κ-casein that is negatively charged and responsible for the electrostatic repulsion of the micelles. The remaining portion of the micelle is called paracasein. Paracaseins thus have increased hydrophobicity that allow them to link with one another by hydrophobic liaisons, which leads to coagulation.

Polysaccharides precipitation. When mixing a protein solution with polysaccharides, one can either observe an incompatibility between the proteins and polysaccharides, a co-solubility of the individual components, or a complexation between the two components. Experimental studies have demonstrated that chitosan, pectins and guaran induce the precipitation of caseins.

Ethanol precipitation. A concentration of 40% ethanol can lead to the precipitation of caseins. Lesser concentrations of ethanol can be used, but the pH must be acidified. This method is rarely used in laboratory or in industries for the precipitation of caseins since it induces protein denaturation, it is not casein-specific, and residual ethanol must be removed following the method.

Calcium chloride precipitation. The addition of calcium chloride at a concentration of 0.2M induces the precipitation of caseins. This method is also rarely used in industries since it is not casein-specific and salt excess must be removed following the method.

Microfiltration. The technique of membrane separation is a logical choice for fractioning milk components because many milk components can be fractionated with regards to their size. Casein micelles can theoretically be isolated by microfiltration according to their hydrolyzed size. Using a microfiltration membrane having 0.1 μm pores with colostrum has been shown to allow for a specific separation of micellar caseins from serum phase. The resulting liquid, called serocolostrum, is limpid, i.e. it does not contains any blood cell, somatic cell, fat particle or casein micelle.

Cryoprecipitation. Caseins can be destabilized and precipitated by cooling the milk at a temperature of −10° C. However, cryoprecipitation has no specific advantages other than to offer alternatives to conventional precipitation methods.

Even with the techniques already known in the art, most of them addressing the precipitation of caseins, it is still highly desirable to be provided with a rapid, easy and inexpensive method for specifically extracting TGF-β and other proteins from milk.

### BRIEF SUMMARY OF THE INVENTION

One aspect of the present invention is to provide a method for obtaining a dairy product supernatant fraction enriched in TGF-β by using specific anionic polysaccharides.

Another aspect of the present invention is to provide a method for obtaining a dairy product precipitate fraction enriched in at least one of TGF-β and lactoferrin by using specific anionic polysaccharides.

Another aspect of the present invention is to provide a method for obtaining a fraction enriched in TGF-β, wherein the method comprises the steps of:
a) adding pectin at a concentration of between 0.01 to 10% w/vol to a liquid dairy product;
b) mixing the dairy product of step a) to induce the formation of a precipitate fraction and a supernatant fraction;
c) allowing for the separation of the precipitate fraction of step b) from the supernatant fraction; and
d) collecting the supernatant fraction as the fraction enriched in TGF-β.

Another aspect of the present invention is to provide a method for obtaining a fraction enriched in at least one of TGF-β and lactoferrin, wherein the method comprises the steps of:
a) adding lambda-carrageenan (λ-carrageenan) at a concentration of between 0.001 to 10% w/vol to a liquid dairy product;
b) mixing the dairy product of step a) to induce the formation of a precipitate fraction and a supernatant fraction;
c) allowing for the separation of the precipitate fraction of step b) from the supernatant fraction; and
d) collecting the precipitate fraction as the fraction enriched in at least one of TGF-β and lactoferrin.

In accordance with the present invention, the term “dairy product” as used herein is intended to encompass milk, including raw milk, whole milk, partially or totally skimmed milk, colostrum and whey. Milk derivatives and transformed product are also meant to be included in the term “dairy product” as they are known in the art to contain at least one of TGF-β and lactoferrin.

In further accordance with the present invention, the term “fraction” as used herein is intended to represent any resulting portion of a whole following a separation process. Usually, the term “fraction” as used herein represents either the precipitate or the supernatant following the separation of a dairy product in accordance with the present invention. The precipitate is usually a substantially solid phase obtained after a settling, a precipitation and/or a centrifugation, while the supernatant is the substantially liquid phase, which can contain soluble proteins, obtained by the same processes as the precipitate.

In yet further accordance with the present invention, the expression “enriched” as used herein is intended to reflect any increase in the quantity and/or concentration of a molecule or a substance following a process when compared with the original quantity and/or concentration of that molecule or substance before the application of the process.

In still further accordance with the present invention, the terms “transforming growth factor-β” and “TGF-β” as used herein are intended to include all known isoforms of TGF-β, particularly TGF-β1, TGF-β2 and TGF-β3. In still further accordance with the present invention, the term “serum albumin” as used herein is intended to include the hydrosoluble albumin proteins serving as transport proteins as known in the art, and is intended to be reflective of the species, including human, from which the dairy product originates from, such as, for example, goat serum albumin. The expression “bovine serum albumin” (BSA) is intended to encompass serum albumin of bovine origins.

In still further accordance with the present invention, the expressions “fractioning”, fractionation”, “separating” and “separation” as used herein interchangeably are intended to represent any mean, process or technique known in the art to fractionate or separate a sample into fractions, such as a supernatant and a precipitate. Those can include, without being restricted to, settling, centrifugation, filtration, microfiltration, pressurization, complexation, dialysis and precipitation such as salt precipitation.

In still further accordance with the present invention, the expressions “collecting” and “collection” as used herein are intended to represent any mean, process or technique known in the art to gather a sample, a determined portion of a sample or a whole of a fraction in liquid or solid form, such as a precipitate or a supernatant. Those can include, without being restricted to, aspiration, pouring, evaporation, drying, lyophilization and desiccation. In the case of a precipitate, it can also involve the resuspension of the precipitate in any type and volume of solvent, including water, following the removal of the supernatant.

In accordance with the present invention, the term “pectin” as used herein is intended to represent the water-soluble colloidal carbohydrate derived from the cell wall of higher terrestrial plants as known in the art, as well as natural or synthetic equivalents, mimetics or derivatives capable of performing the same effect as the one intended for pectin in accordance with the present invention. Such effect include, without being limited to, the phase separation of milk casein micelles by adsorption and/or depletion-flocculation, and the selective precipitation of milk proteins while leaving TGF-β in the supernatant.

In further accordance with the present invention, the terms “carrageenans”, “lambda-carrageenan” and “λ-carrageenan” as used herein are intended to represent the colloidal extract from carrageen seaweed and other red algae as known in the art, as well as natural or synthetic equivalents, mimetics or derivatives capable of performing the same effect as the one intended for pectin in accordance with the present invention. Such effect include, without being limited to, the phase separation of milk casein micelles by adsorption and/or depletion-flocculation, and the selective precipitation of milk proteins including at least one of TGF-β and lactoferrin. The terms “lambda-carrageenan” and “λ-carrageenan” represent a specific subtype of carrageenans as known in the art.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the effects of pectin concentration on remaining protein concentration and pH (indicated on the figure) of the colostrum supernatants fractions. Protein concentration for colostrum solutions with 5% commercial colostrum (), 5% commercial colostrum with pH adjustment to initial pH of colostrum solutions (□) and 10% commercial colostrum (▴);

FIG. 2 illustrates the effects of λ-carrageenan concentration on remaining protein concentration and pH (indicated on the figure) of the colostrum supernatants fractions. Protein concentration for colostrum solutions with 5% commercial colostrum (), 5% commercial colostrum with pH adjustment to initial pH of colostrum solutions (□) and 10% commercial colostrum (▴);

FIG. 3 illustrates the effects of pectin concentration on remaining TGF-β2 concentration of the colostrum supernatants fractions. Protein concentration for colostrum solutions with 5% commercial colostrum (), 5% commercial colostrum with pH adjustment to initial pH of colostrum solutions (□) and 10% commercial colostrum (▴);

FIG. 4 illustrates the effects of λ-carrageenan concentration on remaining TGF-132 concentration of the colostrum supernatants fractions. Protein concentration for colostrum solutions with 5% commercial colostrum (), 5% commercial colostrum with pH adjustment to initial pH of colostrum solutions (□) and 10% commercial colostrum (▴);

FIG. 5 illustrates different approaches for the pilot-scale preparation of growth factors-enriched ingredients using polysaccharides, compared to a reference approach obtained with the casein acid precipitation; and

FIG. 6 illustrates the results of a 2D gels electrophoresis of a commercial colostrum (A), a serocolostrum obtained with λ-carrageenan 0.08% w/vol (B), precipitate casein enriched obtained with λ-carrageenan 0.08% w/vol (C) and a serocolostrum obtained with pectin 0.6% w/vol (D).

### DETAILED DESCRIPTION OF THE PREFERRED EMBODIMENT

In accordance with the present invention, there is provided a new method for the selective fractionation of growth factors from dairy products, such as milk, whey or colostrum, using anionic polysaccharides complexation. It was suggested in the scientific literature that anionic polysaccharides, such as carrageenans and pectin, could interact with bovine milk casein micelles. This principle has been tested for the removal of caseins from bovine colostrum. It should however be noted that the present invention is not specific to bovine dairy products, but to dairy products from any animal source such as cow, sheep, goat, horse, donkey, camel, yak, beaver, water buffalo, reindeer, etc, as will be understood by the skilled man in the art.

In accordance with an aspect of the present invention, the dairy product can be either one of milk, whey or colostrum. In further accordance with the present invention, the growth factor can be TGF-β, and further TGF-β2. In yet further accordance with the present invention, the TGF-β is preferably biologically active. The expression “biologically active” as used herein is intended to encompass an activity or effect conferred to TGF-β on a biological system when the TGF-β is introduced into the biological system.

In accordance with another aspect of the present invention, the anionic polysaccharide can be added to the dairy product at a concentration of between 0.001 to 10% w/vol, preferably of between 0.01 to 10% w/vol, more preferably of between 0.1 to 10% w/vol, and even preferably of between 0.1 to 1% w/vol.

In further accordance with the present invention, the anionic polysaccharide can be pectin, which can be added to the dairy product at a concentration of between 0.01 to 10% w/vol, preferably of between 0.1 to 10% w/vol, more preferably of between 0.1 to 5% w/vol, and even more preferably of between 0.1 to 1% w/vol.

In further accordance with the present invention, the anionic polysaccharide can be carrageenan (including λ-carrageenan), which can be added to the dairy product at a concentration of between 0.001 to 10% w/vol, more preferably of between 0.01 to 10% w/vol, more preferably of between 0.01 to 5% w/vol, even more preferably of between 0.01 to 1% w/vol, and even more preferably of between 0.01 to 0.1% w/vol.

The present invention will be more readily understood by referring to the following examples which are given to illustrate the invention rather than to limit its scope.

### EXAMPLES

### Example I

### Removal of Caseins from Colostrum

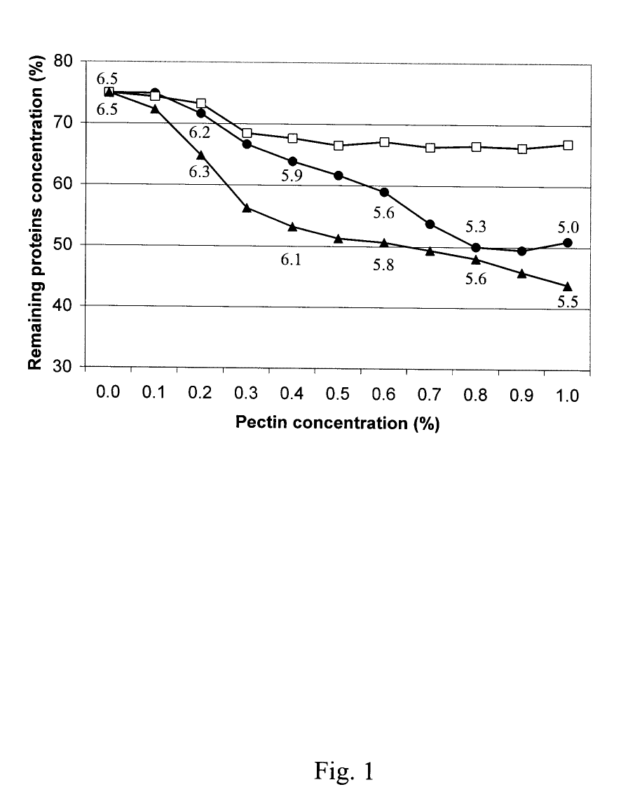

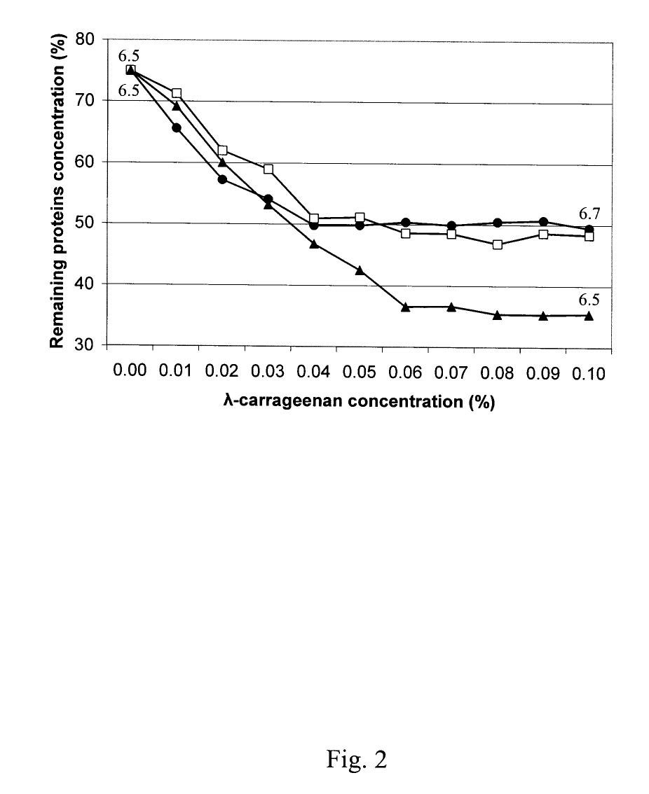

A commercial dried skimmed bovine colostrum, Prodiet™ F520, was rehydrated in order to obtain two solutions with final concentrations of 5% and 10% w/vol of rehydrated colostrum. Each solution was stirred for 2 hours at 4° C. and then mixed with either pectin (final concentration of 0.1 to 1% w/vol) or lambda-carrageenan (final concentration of 0.01% to 0.1% w/vol). Mixing of the dairy product with anionic polysaccharides such as pectin or lambda-carrageenan for example can be performed by any technique known to produced a mixed solution, such as, without being limited to, stirring. The duration and temperature of mixing can be variables as known in the art. The mixed solution can be settled for a determined period of time to allow for the formation of a precipitate and a supernatant. Alternatively, the formation of a precipitate and supernatant can occur during the mixing step. Preferably, the formation of a precipitate can be assessed by direct visualization of the mixed solution. Samples were centrifuged at 12,000 g during 15 minutes. Supernatants were collected and freeze-dried before being analyzed to determine protein concentration. For the highest pectin concentrations (≧0.6% w/vol), protein precipitation was optimal and pH solutions decreased from 6.5 (initial pH) to 5.3 (FIG. 1). The optimal protein precipitation was obtained with lambda-carrageenan concentration of 0.04% w/vol. A higher protein precipitation with lambda-carrageenan was observed when the initial protein concentration of colostrum solution was doubled (FIG. 2).

### Example 2

### Fractionation of Growth Factors

It is known that λ-carrageenan (added at a concentration of 0.08% (% solids)) and pectin (added at a concentration of 0.3% (% solids)) can be used to precipitate caseins from bovine colostrum and accordingly generate a serocolostrum (serum phase of colostrum). Unexpectedly, an examination of the changes in TGF-β2 content of colostrum following casein precipitation, according to precipitation parameters known in the art, showed that pectin generated a serocolostrum rich in TGF-β2 while λ-carrageenan generated a serocolostrum almost free of TGF-β2. Only 10% of the initial TGF-β2 content was found in serocolostrum obtained from treatment with λ-carrageenan. It thus appears that milk growth factors were precipitated in the casein-rich fraction generated from the λ-carrageenan precipitation

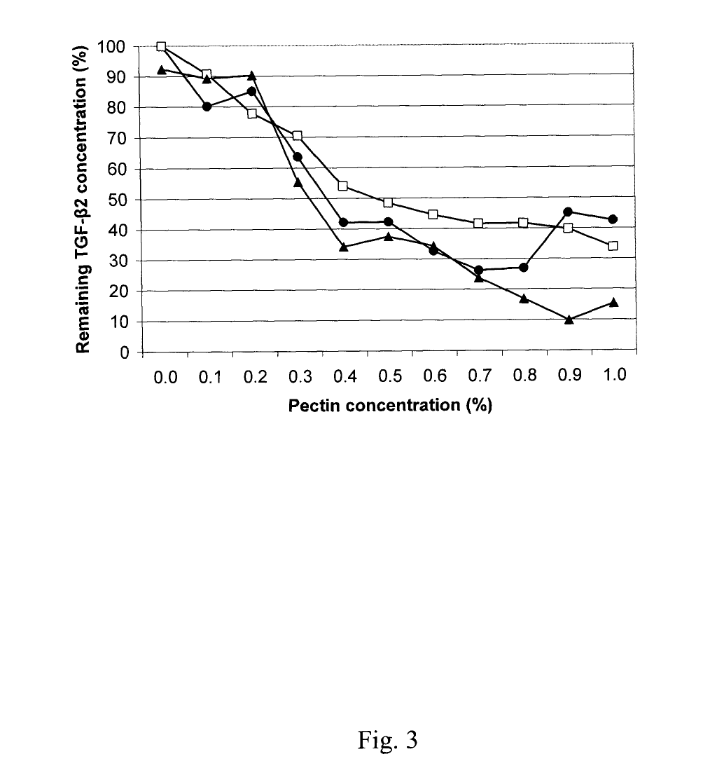

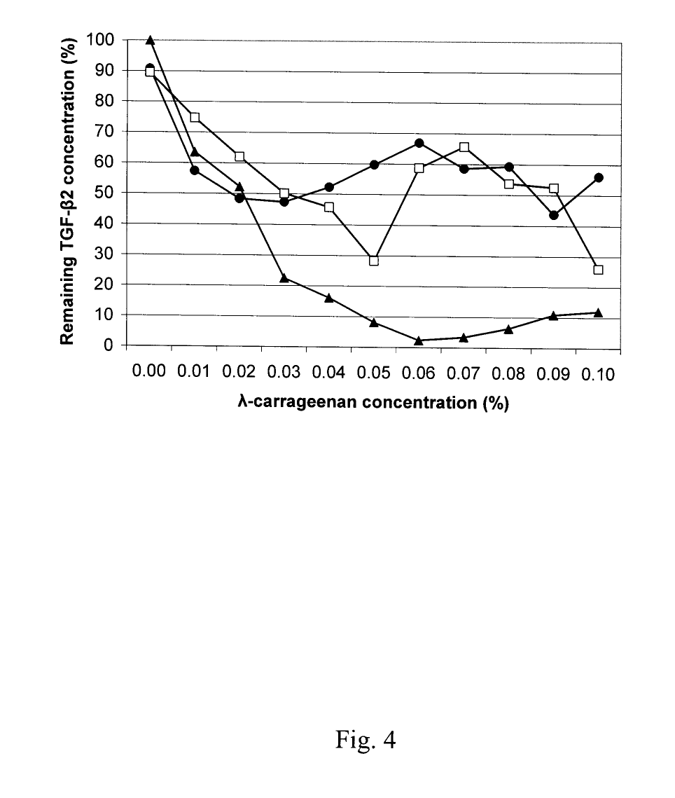

The same experiment as in example 1 was performed, but TGF-β2 precipitation was monitored. It was observed that remaining TGF-β2 concentration decreased with the increase in pectin concentration. Moreover, there were no significant differences between treatments for the TGF-β2 precipitation except for the highest pectin concentration (≧0.6% w/vol) (FIG. 3). For the samples mixed with lambda-carrageenan, remaining TGF-132 concentration decreased with the increase in lambda-carrageenan concentration. Moreover, pH had no influence on TGF-β2 precipitation. Differences were however observed because the system was not in a state of balance with a 5% w/vol initial protein concentration. Initial protein concentration had an important impact on TGF-β2 precipitation. For a 10% w/vol initial protein concentration, the system was in a state of balance and remaining TGF-β2 concentration was close to zero for the highest lambda-carrageenan concentrations (≧0.05% w/vol) (FIG. 4).

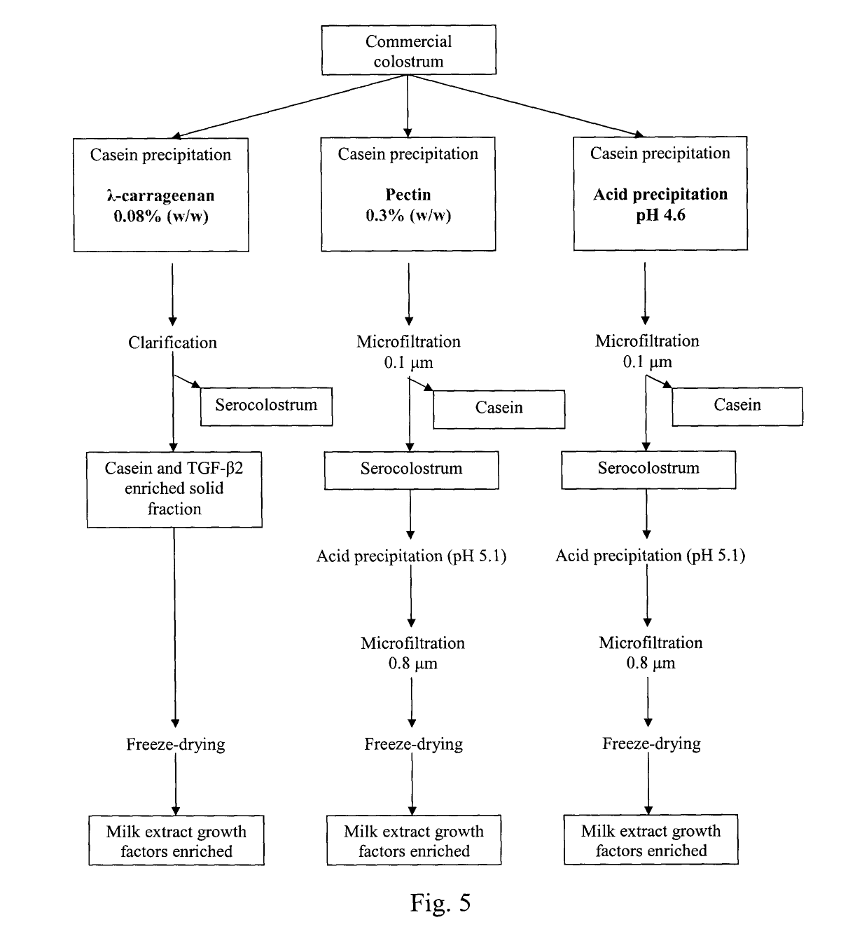

Therefore two different approaches can be used for the preparation of growth factors-enriched fractions (FIG. 5):
The pectin process: This process yielded a supernatant enriched in growth factors, particularly in TGF-β. The supernatants (serocolostrum) recovered upon casein precipitation using pectin can therefore be used directly to perform acid-precipitation of growth factors. Our first experiments have shown that a pH value of 5.1 was optimal for that purpose. This pH value was selected for pilot-scale preparation of prototype samples of growth factors extracts using the pectin process.
The λ-carrageenan process: This process yielded a precipitate enriched in growth factors, particularly in TGF-β and lactoferrin. The λ-carrageenan process therefore mainly consists in separating the material precipitated from colostrum.

These two approaches have been compared to a reference process consisting in a simple isoelectric precipitation (pH 4.6) as depicted in FIG. 5. The pilot-scale experiments were performed with 20-30L colostrum (Prodiet™ F520) rehydrated at 5% w/vol. The complete mass balance of the processes was not performed since the experiments only aimed at generating samples for analytical purposes.

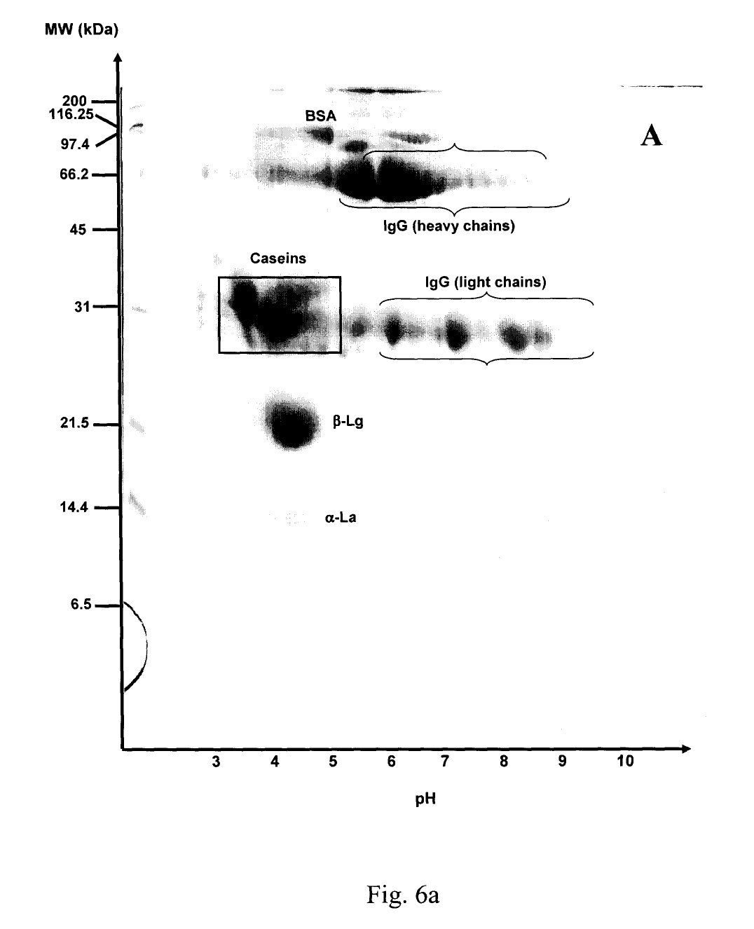

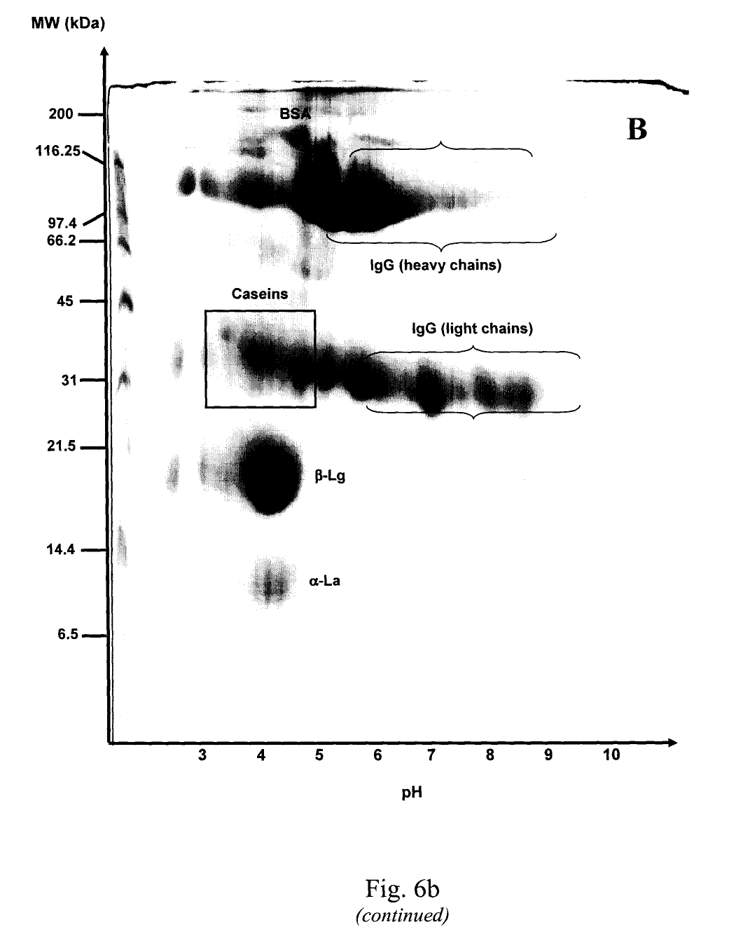

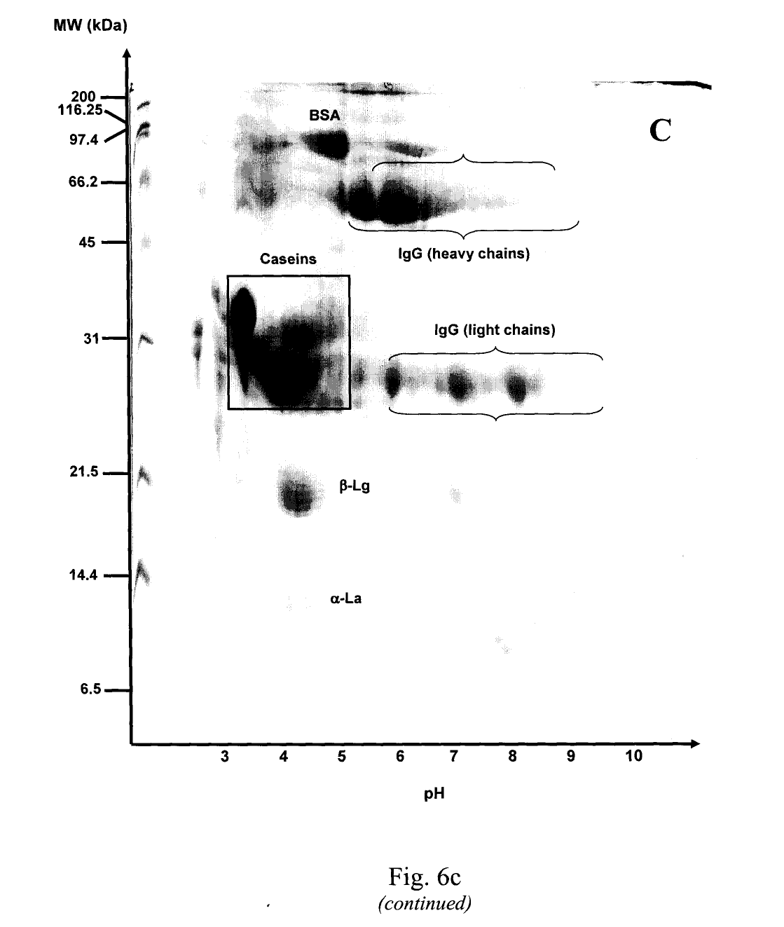

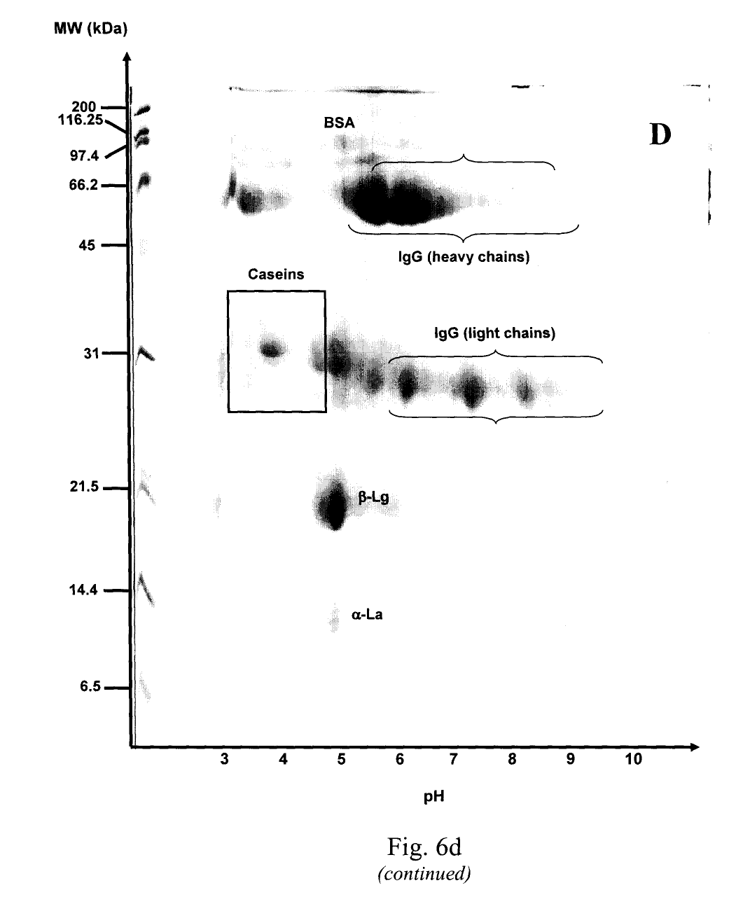

The samples were characterized using two-dimensional (2D) gel electrophoresis in order to highlight differences in the protein and growth factors composition generated by the precipitation processes. The samples tested were a colostrum (as a reference sample, FIG. 6A); a serocolostrum obtained with λ-carrageenan at 0.08% w/vol (FIG. 6B); a casein-rich precipitate obtained with λ-carrageenan at 0.08% w/vol (FIG. 6C); and a serocolostrum obtained with pectin at 0.6% w/vol (FIG. 6D).

Colostrum: FIG. 6A shows strong bands (or spots) corresponding to caseins and immunoglobulins. Only very faint bands were detected at alkaline pH and low molecular weight where growth factors were expected to appear. These bands are consisting of specific peptides from caseins and other minor proteins.

Serocolostrum λ-carrageenan: FIG. 6B shows that an important quantity of colostrum caseins are missing from the serocolostrum. There was whey protein (β-lactoglobulin, α-lactalbumin) and immunoglobulin enrichment. No bands were found in the alkaline pH and low molecular weight zone.

Precipitate λ-carrageenan: FIG. 6C shows that there was a casein enrichment in the fraction precipitated with λ-carrageenan. There was also some whey proteins and IgG in this fraction. A larger number of bands was found in the alkaline pH and low molecular weight zone.

Serocolostrum pectin: FIG. 6D shows that almost all colostral caseins disappeared from the serocolostrum. Compared to initial colostrum (FIG. 6A), there were no whey proteins or immunoglobulin enrichment. Very few bands were visible in the alkaline pH and low molecular weight zone.

With the observation of two-dimensional gels, we concluded that λ-carrageenan and pectin can generate serocolostrum by precipitating the major part of colostrum caseins. Moreover, those results show that the precipitated fractions obtained with the λ-carrageenan treatment are enriched with some specific peptides and other minor proteins. However, the serocolostrum obtained with the pectin treatment is not enriched in these specific peptides and minor proteins compared to initial colostrum.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for obtaining a fraction enriched in transforming growth factor-β, wherein the method comprises the steps of
a) adding pectin at a concentration of between 0.01 to 10% w/vol to a liquid dairy product;
b) mixing the dairy product of step a) to induce the formation of a precipitate fraction and a supernatant fraction;
c) allowing for the separation of the precipitate fraction of step b) from the supernatant fraction; and
d) collecting the supernatant fraction of step c) as the fraction enriched in transforming growth factor-β.

2. The method of claim 1 wherein the liquid dairy product is selected from the group consisting of milk, whey or colostrum.

3. The method of claim 1 wherein the separation of the precipitate fraction from the supernatant in step c) is performed by any one of: settling, centrifugation, filtration, microfiltration, pressurization, complexation and dialysis.

4. The method of claim 1 wherein the transforming growth factor-β is transforming growth factor-β2.

5. The method of claim 1 wherein the concentration of pectin is between 0.1 to 10% w/vol.

6. The method of claim 1 wherein the concentration of pectin is between 0.1 to 1% w/vol.

7. A method for obtaining a fraction enriched in at least one of transforming growth factor-β and lactoferrin, wherein the method comprises the steps of
a) adding lambda-carrageenan at a concentration of between 0.001 to 10% w/vol to a liquid dairy product;
b) mixing the dairy product of step a) to induce the formation of a precipitate fraction and a supernatant fraction;
c) separating the allowing for the separation of the precipitate fraction of step b) from the supernatant fraction; and
d) collecting the precipitate fraction of step c) as the fraction enriched in at least one of transforming growth factor-β and lactoferrin.

8. The method of claim 7 wherein the liquid dairy product is selected from the group consisting of milk, whey or colostrum.

9. The method of claim 7 wherein the separation of the precipitate fraction from the supernatant in step c) is performed by any one of: settling, centrifugation, filtration, microfiltration, pressurization, complexation and dialysis.

10. The method of claim 7 wherein the transforming growth factor-(3 is transforming growth factor-β2.

11. The method of claim 7 wherein the concentration of lambda-carrageenan is between 0.01 to 10% w/vol.

12. The method of claim 7 wherein the concentration of lambda-carrageenan is between 0.01 to 0.1% w/vol.

13. The method of claim 7 wherein the precipitate fraction is further enriched in serum albumin.

14. The method of claim 13 wherein the serum albumin is bovine serum albumin.
